(19) United States
(12) Patent Application Publication
(10) Pub. No.: US 2014/0121730 A1
(43) Pub. Date: May 1, 2014

(54) SYSTEM AND METHOD FOR FRACTIONAL SCANNER AND TREATMENT

(71) Applicant: LUMENIS LTD., Yokneam Ilit (IL)

(72) Inventors: Reuven KALEV, Elyachin (IL); Oren LIAV, D.N. Misgav (IL)

(73) Assignee: LUMENIS LTD., Yokneam Ilit (IL)

(21) Appl. No.: 14/068,974

(22) Filed: Oct. 31, 2013

Related U.S. Application Data
(60) Provisional application No. 61/720,789, filed on Oct. 31, 2012.

(51) Int. Cl. A61N 5/06

(52) U.S. Cl. CPC A61N 5/0613
USPC 607/89

(57) ABSTRACT

A method and apparatus for treating tissue includes an energy application device that directs energy at the skin tissue of the patient in a fractional treatment mode in which a plurality of spots receive energy which may be in a form of a laser beam. In order to lessen thermal damage to the surrounding tissue of the spot, a controller will cause the laser or the energy application device to skip around the area to be treated rather than to apply the laser beam in a sequential, raster-like fashion to each of the points in a fractional treatment area.

### RELATED APPLICATIONS

This application relates to and claims priority to U.S. application Ser. No. 61/720789, filed Oct. 31, 2012, the entirety of which is herein incorporated by reference.

### FIELD OF THE INVENTION

This application is in the field of laser-based skin resurfacing apparatus and methods and relates to the lessening of skin overheating and skin damage during a so-called fractional treatment of the human skin tissue.

### COMPUTER LISTING

Attached to and forming a portion of the present specification is a computer program listing containing an exemplary listing of programming which may be used in connection with the method and apparatus of the present invention. The computer program listing is a copyrighted work owned by the assignee of the present invention and a copyright notice has been applied to the listing.

### BACKGROUND

Prior art systems and methods include apparatus and methodologies for providing so-called fractional treatment to the human skin tissue using one of laser or other energy providing devices. These prior art systems may include systems and methods for providing one of or both of so-called ablative treatment and so-called non-ablative treatment.

In a typical prior art fractional treatment system, a number of treatment spots within a defined area of the human skin are made. These may be few as 25 going up to over 1000 treatment spots in a given defined area of the human skin tissue. The treatment spots may be delivered using a laser device which directs the laser beam in a so-called “scan” mode in a known manner. The treatment spots may be rectangular, square, circular or any shape that the laser is capable of making. Examples of prior art laser scanning devices are U.S. Pat. Nos. 5,743,902, 5,938,657, 5,957,915, and 6,328,733, the entireties of which are incorporated herein by reference. In addition, fractional treatment systems and methods are disclosed in co-pending application Ser. No. 13/038773, filed Mar. 2, 2011, as well as application Ser. No. 13/314548, filed Dec. 8, 2011, the entireties of which are also incorporated herein by reference.

In the prior art systems for providing fractional treatment using a scanning laser device, it is known to provide a laser beam “shot” sequentially, that is, in a given matrix of, for example, a 5×5 square, sequentially providing a laser beam application in a raster like format. This is illustrated in FIG. 1, labeled “prior art”. A problem with a raster like format is that there is a likelihood that the surrounding tissue may overheat, causing discomfort to the patient and possibly causing excess damage to the skin tissue and may even result in a non-fractional treatment due to bulk heating of the whole skin tissue. Ideally, it would be desirable to wait until the skin surface to the previously made “shot” cools off before providing a “shot” to the neighboring skin tissue. The concept of “thermal relaxation time” exists and is generally understood to be the time it takes for the tissue cool down to normal tissue temperature after having been provided with a laser “shot”. The thermal relaxation time varies somewhere between 50-800 msec depending on the skin type, the laser device, the area of treatment and the laser penetration level and energy. However, in a raster scan type of laser application, waiting for the thermal relaxation time before applying and next “shot” to the neighboring skin tissue would cause the treatment to extend over a long amount of time which is unacceptable in present treatment applications.

Thus, there is a need for an apparatus and method which avoids the deficiencies described above and yet provides sufficient fractional treatment to be efficacious for the patient.

### SUMMARY

In one aspect, a method is provided for fractional treatment to a skin tissue, in which the method comprises the steps of providing a laser apparatus for applying laser beam energy to the skin tissue, then selecting, on the laser apparatus, a matrix of laser beam energy application to the skin tissue having one or more of the attributes of: shape, size, energy and the density of application spots to the matrix on the skin tissue. Then the laser apparatus is activated in a manner so as to eliminate or at least minimize adjacent application spots sequentially receiving laser beam energy.

In another aspect, the matrix is in the form of an X by Y format and the application of laser energy is in a raster like format.

In yet another aspect, after an application spot has received laser energy, the laser apparatus skips N number of application spots prior to activating the next laser beam application.

Further, a system for providing fractional treatment to a skin tissue is disclosed. A laser device directs laser energy at the skin tissue of the patient. The laser device includes a controller to select a matrix of laser application spots on the skin tissue. The matrix may be selected to have one or more of the following attributes, including shape, size, energy and the density of application points to the matrix on the skin tissue. A controller controls the application of laser application spots so as to eliminate or at least minimize adjacent application spots sequentially receiving laser beam energy.

### BRIEF DESCRIPTION OF THE DRAWINGS

FIG. 1 is an illustration of a prior art apparatus and method for fractional treatment.

FIGS. 2a-2c are illustrations of a fractional treatment embodying the inventions of the present application in a 5×5 matrix.

FIG. 3 illustrates the sequence of laser beam application for a 9×9 matrix,

FIG. 4 illustrates an application of the present invention using a spiral-shaped scanning technique.

### DETAILED DESCRIPTION

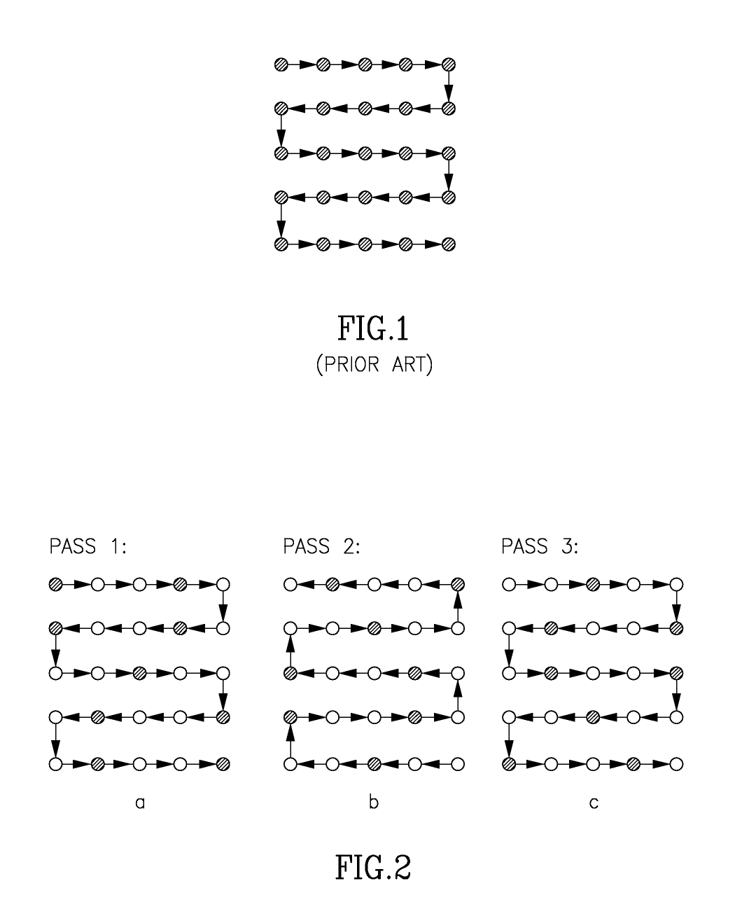

Turning now to FIG. 1, this figure illustrates the prior art methodology of fractional treatment using a scanning laser in a raster like application. As can be seen from the drawing figure, the laser is applied in a raster like fashion across one row in the area of treatment, then down one level and across another row of application points. This may cause, due to the proximity of the treatment spots to each other sequentially, the skin to warm up in an undesirable manner, as discussed above.

Turning now to FIGS. 2a-2c, these figures illustrate, in an exemplary 5×5 area of treatment, the operation and sequence of application of a laser to the skin tissue. It is to be understood that the 5×5 matrix is by way of example only, as any size or shape of a surface area treatment may be utilized. In FIG. 2a, noted as “Pass 1”, the dark circles illustrate the sequence of laser beam application to the skin tissue in this first pass. After the first pass, as illustrated in FIG. 2b, the laser beam again applies beams to other portions of the skin tissue without repeating an application on the same spot previously made in pass 1. Finally, in pass 3, illustrated FIG. 2c, the remaining spots that have not been previously had laser application applied to them have a laser beam applied. It can be seen by a review of FIGS. 2a-2c that no one point in the matrix had a laser beam applied more than once during a first scan. The sequence of the application of laser beams as shown in FIGS. 2a-2c is for the purpose of illustration only, since other sequences of laser beam application may be made, so long as in the initial passes, a spot is not subject more than once to a laser beam application. In subsequent passes, the same spot, as desired, may be “hit” again (and again). However, it may be that adjacent spots may be hit. For example, in FIG. 2a, it can be seen that spots (solid circles) 2 and 3 in the sequence may be considered adjacent. In this case, it can be further seen that the adjacent spots 1 and 4 are towards the “edge” of the matrix and will likely not cause overheating of the skin tissue because the area to right (in FIG. 2a) is an area of skin tissue outside the matrix and thus not being subjected to laser energy beams.

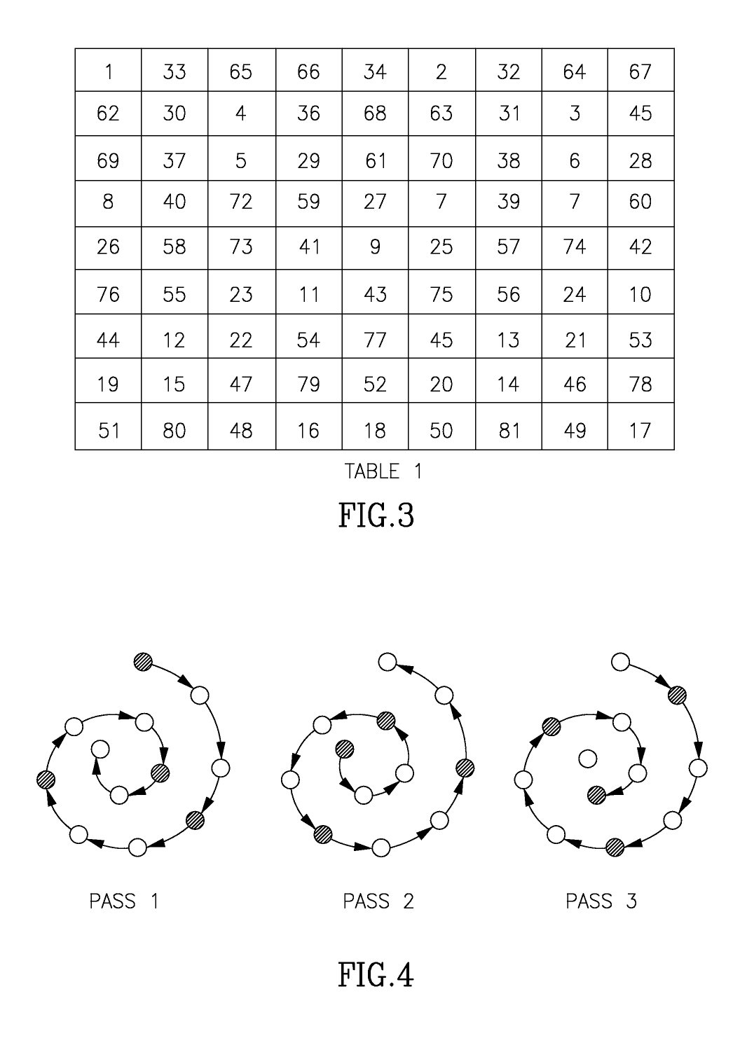

Turning now to FIG. 3, that figure illustrates the application of laser beam energy to a 9×9 matrix of application points. In the embodiment of FIGS. 2a-2c there is illustrated a 5×5 matrix in which three passes are required to ‘hit’ each of the 25 spots, and this is done by hitting a spot, skipping two spots and hitting the third spot. In FIG. 3, the 9×9 matrix has a total of 81 application points. With this number of application points, a first spot is “hit”, the next four spots skipped, and the fifth spot “hit”. This sequence has five passes in order to apply laser energy to each of the 81 spots. Of course, the discussion of a 5×5 matrix or a 9×9 matrix is for the purposes of illustration only, and any size or shape matrix and any number of spots or passes may be provided in the present invention's scope.

Turning now to FIG. 4, that figure illustrates a scanning mode in which the scanning is in a spiral-like shape. U.S. patent application Ser. No. 13/038,773, filed Mar. 2, 2011, the entirety of which is herein incorporated by reference, illustrates scanning in a spiral-shape (see FIG. 6A therein, for example) as well as other shapes (see FIGS. 8A-8D, for example). The “skipping” feature of the present invention may be applied to any scanning shape, whether it be square-shaped, as in FIG. 2, spiral-shaped as in FIG. 4, or any shape that the laser apparatus is capable of making. In FIG. 4, it can be seen that each spot is not “hit” during an initial pass of the laser device, but rather spots are skipped so as to lessen the damage to the skin surface yet provide total coverage of the area scanned.

The present invention allows the laser operator to select the size of the area to be scanned on the skin surface as well as a spot density. Based upon the foregoing, the distance between the spots is determined. In addition, the operator can adjust the laser device parameters, such as the time duration of each laser pulse, spot size and the laser energy per pulse. Also, in the present invention, the operator may select the number of passes that the scanner needs to pass in order to apply laser energy to all spots in the pattern chosen. In the pattern shown in FIG. 2, three passes are shown in order to ensure that all spots are lased. While the figure illustrates three passes, any number of passes may be chosen as selected by the operator. However, the number of passes selected determines how many spots will be skipped from a laser application spot to the next laser application spot. Thus, in FIG. 2, since three passes were chosen for a 5×5 matrix the scanner will, after laser energy is applied to a selected spot, skip two spots and apply laser energy to the third spot. It is to be understood that the “spots” referred to are not physical spots on the skin tissue, but rather laser application points as illustrated in the drawing figures. In addition, the operator may desire, even after the multiple passes of FIGS. 2-4, not to hit each and every spot on the skin tissue as the treatment formulation encompasses.

As can be seen in reference to FIGS. 2a-c, the multi pass aspect may be seen as lengthening the amount of time required to make one complete pass of a given spot matrix simply because more than one pass may be required. However, this is somewhat ameliorated due to the fact that the time to move the laser beam from, for example, spots 1 and 4 in FIG. 2a may be less than the time to hit spot 1, then move the beam and hit spot 2 and then hit spot 3, etc., due to the time needed to move the laser beam from spot to spot. Even if there is a somewhat more lengthy time to complete a complete scan of all spots than the prior art method of FIG. 1, this is made up by the avoidance of overheating of the skin tissue.

Also, in an additional embodiment of the present invention, the system may allow and provide different types of pulses in each of the passes. For example, the operator may cause a controller to make a first pass of ablative energy which will form micro channels of a selected depth and width and then during the second pass or other subsequent pass may provide non-ablative energy application to that same spot or other spots. In this way, the system may create different combinations and distributions of treatments to obtain different tissue effects as desired by the operator.

As a part of the present specification, a computer program listing appendix has been included which illustrates one exemplary fashion of implementing the present invention. This computer program listing is written in C++ computer program language and may be implemented on a PC or other computer systems.

The present invention may be incorporated into a fractional treatment laser device which is programmable to provide fractional treatment of selected spots in different selected patterns, energy levels, etc. One such system is the M22 system which is available from Lumenis Ltd, of Yokneam, Israel, and the sequence of laser energy application described herein may be incorporated in a suitable computer software program or set of instructions on the M22 system or any other suitable system. The laser energy for the non-ablative laser treatment may be for example a YAG laser, a 1565 nm fiber laser, but may be of any other suitable wavelength device. The M22 system can, by way of example, encompass a 15 W laser which can deliver, for a 1 msec pulse width, 15 mJ of energy, or for a 5 msec pulse width, deliver 75 mJ of energy. The desire and aim in fractional treatment by laser beams is to deliver the desired energy to the skin tissue in the shortest amount of time so as to both provide effective treatment and reduce discomfort to the patient. The M22 may be programmed in a known manner to create a “skipping” sequence once the operator has inputted the number of spots, the density of the spots, the pulse width, etc. in an interactive manner. Alternatively, the M22 may contain one or more preprogrammed lookup tables to allow the operator to select a particular treatment from a list of available procedures. The M22 system is also available with an active cooling system contained in a tip of a handpiece that contacts the skin. Such cooling tips are known in the art. However, combining such a cooling tip with the “skipping” sequence as described herein allows further avoidance of overheating the skin tissue and permits the operator to safely apply more energy to each pulse than would be possible with the prior art of the type exemplified by FIG. 1, even if the embodiment of FIG. used a cooling tip.

The present invention, which may be termed a “multipass” device, may, as discussed above, be used to non-randomly apply laser application points so as to avoid adjacent laser application points close in time and avoid applying laser energy to the same spot more than once. However, it may be desirable to apply laser energy to the same spots more than once if further treatment is deemed necessary.

While in the above discussion the fractional treatment is generally of the non-ablative type, the present invention may be applied to ablative fractional treatment. During known ablative fractional treatment, it may be necessary, once a channel has been formed in the skin surface in a known manner, to once again apply a laser beam to that same channel in order to deepen the depth of the channel or to provide a non-ablative energy to the bottom of the channel. In prior art systems, there exists the possibility of causing excess heat to be applied to the skin tissue, to the discomfort of the patient and possible unwanted thermal damage. With the present invention, the non-sequential application of laser beams to the surface will lessen discomfort and thermal damage due to the laser beams being applied in a manner such that the skin does not excessively heat up.

Having thus described at least one illustrative aspect of the invention, various alterations, modifications and improvements will readily occur to those skilled in the art. Such alterations, modifications and improvements are intended to be within the scope and spirit of the invention. Accordingly, the foregoing description is by way of example only and is not intended as limiting. The invention's limit is is defined only in the following claims and equivalents thereto.

```
POS * MultiPassAlgorithm ( POS * RasterArr, int ArrSize , int NumofPasses)
{
  POS * MultiPassArr; // the multi pass array
  int RasterArrPos =0;  // hold the raster array position
  int MultiPassPos =0;  // hold the multi pass array position
  int PassNo =0;  // hold the pass number
  bool bUP = true; // flag of the scan direction
  int NextPos;
  int iNumberOfSteps;
  iNumberOfSteps = ArrSize/NumOfPasses ; // calculate the number of steps in each pass
  MultiPassArr = new POS[ArrSize];  // create the multi pass array
  MultiPassArr[0] =RasterArr[0];  // set the first position
  // fill the multi pass array
  for (MuliPassPos=1; MultiPassPos < ArrSize; MultiPassPos++)
  {
    if (bUp)
    {
      // the direction count is up in the array
      RasterArrPos +=NumOfPasses;
      if (RasterArrPos >= ArrSize)
      {
        PassNo++;
        if (PassNo>= NumOfPasses)
        {
          break;
        }
        else
        {
          //calculate the next count
          NextPos = iNumbrtOfSteps * NumOfPasses + PassNo;
          if (NextPos >= ArrSize)
          {
            // out of array , set to - Jump
            NextPos -= NumOfPasses;
          }
        }
        RasterArrPos =NextPos;
        bUp = false;
      }
    }
    else
    {
      // the direction count is down in the array
      RasterArrPos -= NumOfPasses;
      if (RasterArrPos < 0)
      {
        PassNo++;
        if (PassNo>= NumOfPasses)
        {
          break;
        }
        else
        {
          RasterArrPassNo =PassNo;
        }
        bUp = true;
      }
    }
    MultiPassArr[MultiPassPos] =RasterArr[RasterArrPos];
  }
  return MultiPassArr;
}
```

What is claimed is:

1. A method for providing fractional treatment to a skin tissue, the method comprising:
providing a laser apparatus for applying laser beam energy to the skin tissue;
selecting, on the laser apparatus, a matrix of laser beam energy application spots on the skin tissue having one or more of the attributes of shape, size, energy, pulse width and the density of application spots to the matrix on the skin tissue; and,
activating the laser apparatus such that adjacent application spots sequentially receiving laser beam energy is one or more of eliminated or minimized.

2. The method of claim 1, wherein the matrix is in the form of an X by Y format and wherein the application of laser energy is in a raster like format.

3. Method of claim 2, wherein, in the application of laser energy, after an application spot has received laser energy, the laser apparatus skips Y number of application spots on the matrix prior to activating the next laser beam application.

4. A system for providing fractional treatment to a skin tissue, the system comprising:
a laser device to direct laser energy at the skin tissue of the patient;
the laser device including a controller to select a matrix of laser application spots on the skin tissue, wherein the matrix may be selected to have one or more of the following attributes: shape, size, energy, pulse width and the density of application spots to the matrix on the skin tissue;
the controller controlling the application of laser application spots such that adjacent spots sequentially receiving laser beam energy is one or more or eliminated or minimized.
